(19) United States
(12) Patent Application Publication
(10) Pub. No.: US 2024/0389870 A1
(43) Pub. Date: Nov. 28, 2024

(54) Blood Pressure Monitoring System Including a Liquid Filled Sensor

(71) Applicant: Apple Inc., Cupertino, CA (US)

(72) Inventor: Caleb C. Han, San Jose, CA (US)

(21) Appl. No.: 18/593,644

(22) Filed: Mar. 1, 2024

Related U.S. Application Data
(60) Provisional application No. 63/468,094, filed on May 22, 2023.

(51) Int. Cl. A61B 5/022; A61B 5/00; A61B 5/021; A61B 5/0225

(52) U.S. Cl. CPC A61B 5/02225; A61B 5/02133; A61B 5/02208; A61B 5/02233; A61B 5/0225; A61B 5/681; A61B 5/6831; A61B 5/742; A61B 2560/0257; A61B 2560/0462; A61B 2562/0219; A61B 2562/0247; A61B 2562/164; A61B 2562/168

(57) ABSTRACT

Embodiments are directed to a blood pressure measurement device that includes a strap operable to couple the blood pressure measurement device to a user and a pump coupled to the strap. In some embodiments, the blood pressure measurement device includes an inflatable chamber fluidly coupled to the pump and positioned between the pump and the user when the blood pressure measurement device is coupled to the user. The inflatable chamber can be configured to expand towards the user when inflated. The blood pressure measurement device can include a sensing chamber coupled to the inflatable chamber and configured to be placed between the inflatable chamber and the user; a pressure sensor positioned in the sensing chamber and configured to detect a pressure of the liquid; and a vibration sensor positioned in the sensing chamber and configured to detect vibrations due to blood flow of the user.

### CROSS-REFERENCE TO RELATED APPLICATION

This application is a nonprovisional and claims the benefit under 35 U.S.C. § 119 (e) of U.S. Provisional Patent Application No. 63/468,094, filed May 22, 2023, the contents of which are incorporated herein by reference as if fully disclosed herein.

### FIELD

The described embodiments relate generally to blood pressure measurement devices, and more particularly, the described embodiments relate to blood pressure measurement devices including a liquid filled sensor.

### BACKGROUND

A user may monitor one or more of their physiological parameters by attaching a monitoring device such as a blood pressure monitor to one of their limbs. The blood pressure monitor may include a cuff that secures an inflatable bladder against a limb of the user. The bladder can be inflated to compress the limb, thereby compressing one or more blood vessels in the limb, and restricting and/or stopping blood flow through the vessels. The pressure in the bladder may be measured during inflation or deflation and used to determine a blood pressure of the user. This type of blood pressure measurement may be referred to as oscillometry blood pressure measurement.

It can be difficult to precisely determine conventional blood pressure parameters, such as systolic and diastolic blood pressures, using oscillometry measurement techniques. It may be desirable to more precisely determine events associated with a blood pressure measurement, including the closing and/or opening of a blood vessel during a compression measurement.

### SUMMARY

Embodiments are directed to a blood pressure measurement device that includes a strap operable to couple the blood pressure measurement device to a user, a pump coupled to the strap, and an inflatable chamber fluidly coupled to the pump and configured to expand towards the user when inflated. The blood pressure measurement device can include a sensing chamber coupled to the inflatable chamber and configured to be compressed between the inflatable chamber and the user when the blood pressure measurement device is coupled to the user. The sensing chamber can be filled with a liquid. The blood pressure measurement device can include a pressure sensor positioned in the sensing chamber and configured to detect a pressure of the liquid and a vibration sensor positioned in the sensing chamber and configured to detect vibrations due to a blood flow of the user.

Embodiments are also directed to a blood pressure measurement device that includes a strap operable to couple the blood pressure measurement device to a user. The strap can include an inflatable chamber configured to expand toward the user when inflated. The blood pressure measurement device can include a support plate coupled to the strap and positioned between the strap and the user when the blood pressure measurement device is worn by the user and a sensing chamber coupled to the support plate, positioned between the support plate and the user when the blood pressure measurement device is coupled to the user, and filled with a liquid. The blood pressure measurement device can include a pressure sensor positioned in the sensing chamber and configured to detect a pressure of the liquid, and a vibration sensor positioned in the sensing chamber and configured to detect vibrations due to blood flow of the user

Embodiments further include a blood pressure measurement system including a smartwatch, a blood pressure measurement device, and a strap. The smartwatch can include a housing, a display positioned at least partially within the housing, and a processor positioned in the housing. The blood pressure measurement device can include an inflatable chamber configured to expand towards a user when inflated and a sensing chamber coupled to the inflatable chamber and configured to be placed between the inflatable chamber and the user. The sensing chamber can be filled with a liquid. The blood pressure measurement device can include a pressure sensor positioned in the sensing chamber and configured to detect a pressure of the liquid and a vibration sensor positioned in the pressure chamber and configured to detect vibrations due to blood flow of the user. The strap can couple the smartwatch to a first location on the user, couple the blood pressure measurement device to a second location on the user and transmit one or more signals between the smartwatch and the blood pressure measurement device.

### BRIEF DESCRIPTION OF THE DRAWINGS

The disclosure will be readily understood by the following detailed description in conjunction with the accompanying drawings, wherein like reference numerals designate like structural elements, and in which:

FIG. 1A shows an example blood pressure measurement device being worn by a user;

FIG. 1B shows an example cross-sectional view of the example blood pressure measurement device being worn by a user in a first state;

FIG. 1C shows an example cross-sectional view of the example blood pressure measurement device being worn by a user in a second state;

FIG. 2 shows a cross-sectional view of an example blood pressure measurement device;

FIG. 3 shows a cross-sectional view of an example blood pressure measurement device;

FIG. 4 shows a cross-sectional view of an example blood pressure measurement device;

FIG. 5A shows a perspective view of an example blood pressure measurement device;

FIG. 5B shows a cross-sectional view of the example blood pressure measurement device shown in FIG. 5A;

FIG. 6 shows an example process for operating a blood pressure measurement device;

FIG. 7 shows an example process for operating a blood pressure measurement device; and

FIG. 8 shows an example system diagram for a blood pressure measurement device.

It should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications and equivalents as can be included within the spirit and scope of the described embodiments as defined by the appended claims.

Embodiments disclosed herein are directed to a blood pressure measurement device that uses a liquid filled sensing chamber to measure blood pressure of a user. The blood pressure measurement device can include a compression system that is separate from the liquid filled sensing chamber. In these embodiments the sensing chamber can be filled with a fixed amount of liquid and be sealed so that the amount of liquid in the sensing chamber does not vary during a measurement procedure. The compressions system can press the liquid filled sensing chamber against the skin of the user during a blood pressure measurement procedure or blood flow measurement procedure. In some cases, a vibration sensor can be positioned in the liquid filled sensing chamber and measure vibrations transmitted from the user and to the liquid in the sensing chamber. The vibration sensor can be configured to detect vibrations due to blood flow, such as vibration associated with Korotkoff measurements (e.g., opening and closing of a blood vessel). The liquid filled sensing chamber may help transmit vibrations from blood flow (e.g., blood flow sounds) to the vibration sensor, which may allow Korotkoff measurements to be performed by an electronic device.

Additionally or alternatively, the blood pressure measurement devices described herein can be operated to measure vibrations due to blood flow at one or more conditions. For example, the compression system may be inflated until the system determines that a criteria is satisfied (e.g., identifying a maximum sound intensity of blood flow, identifying partial collapse of a blood vessel, etc.). In response to determining that a criteria is satisfied, the blood pressure measurement device may maintain a current inflation condition and measure blood flow sounds over a period of time. The measured blood flow sounds may be used to determine one or more physiological parameters such as heart rate, blood pressure, heart rate variation and so on.

In some cases, the sensing chamber can also include a pressure sensor that is configured to measure pressure of the liquid in the sensing chamber. The liquid filled pressure sensing chamber may be more sensitive to pressure changes in the blood vessel as compared to conventional air filled pressure sensing chambers due to the incompressible nature of liquid. Further, having the vibration sensor and pressure sensor separated from the inflation system may help reduce noise and/or increase accuracy of blood pressure measurements (or other blood flow measurements) as compared to typical blood pressure measurement systems in which a single air inflated cuff is used to both compress a user's arm and measure air pressure in the cuff to estimate blood pressure.

In some embodiments, the blood pressure measurement device is implemented as a pressure sensing stack that includes a liquid filled sensing chamber positioned on an inflatable chamber. A strap can secure the stack against a skin surface of a user such that the sensing chamber is positioned between the inflatable chamber and the user. The inflatable chamber can be inflated to expand towards the user thereby pressing the sensing chamber against the user's skin surface. The sensing chamber can be used to measure vibrations due to blood flow and blood pressure of the user during the inflation and/or deflation of the inflatable chamber. The sensor chamber can include a flexible housing that contains the volume of liquid. In some cases, the stack structure can help decrease the size of the blood pressure measurement device such that it can be integrated into smaller devices while achieving desirable accuracy and reliability.

The blood pressure measurement device can also include a pump that is configured to inflate the inflatable chamber. The pump can be integrated into the pressure sensing stack structure of the pressure sensing device. For example, the inflatable chamber can be positioned on an upper surface of the pump such that when the blood pressure measurement device is worn by the user the stack includes the pump positioned furthest from the user, the inflatable chamber positioned between the pump and the user, and the sensing chamber positioned between the inflatable chamber and the user. The strap can secure the pressure sensing stack to the user such that, as the inflatable chamber is inflated, it expands primarily towards the user.

In some implementations, the blood pressure measurement device includes a support plate that is positioned between the inflatable chamber and the sensing chamber. The support plate can be a rigid or semi-rigid structure that helps distribute the force generated by the inflatable chamber across the sensing chamber and can prevent blood pressure pulse signal loss that may occur between the sensing chamber and the inflatable chamber. In some cases, the inflatable chamber, the support plate, and the sensing chamber can all have similar profiles such that the support plate and/or expansion chamber extend across substantially the entire bottom surface of the sensing chamber.

The blood pressure measurement device can be an independent device that includes a display, dedicated processor, battery, and so on, which can be mounted to or otherwise integrated into the strap. In other cases, the blood pressure measurement device can be integrated to function with a wearable device such as a smartwatch. For example, the pressure sensing stack can be mounted to a smartwatch band and be electrically coupled with the smartwatch. In these cases, the pressure sensing stack could send vibration and/or pressure measurements to the smartwatch and the smartwatch can analyze, display or combine the vibration and pressure data with other data that was collected by the smartwatch, which could include ambient pressure, user movement, other physiological measurements of a user such as electrocardiograms, temperatures, oxygen saturation, and so on. In some cases, the blood pressure measurement device could utilize components of the smartwatch such as a power source, processor, a display or other output devices, communications hardware and so on.

The example of a smartwatch is given as one example of a device that the blood pressure measurement device can be integrated with. However, the blood pressure measurement device can also be configured to detect blood pressure at locations other than the wrist and/or integrated with other devices such as a dedicated display and processing system, a smartphone, other wearable health monitors, portable music players, and the like. For example, the blood pressure measurement device could be mounted to a strap that is configured to wrap around the upper arm, the wrist, and/or other portions of a user to estimate a blood pressure using measurements from one or more of these locations.

In some cases, the blood pressure measurement device can wirelessly communicate with one or more other devices. For example, the pressure sensing device could send blood pressure measurement data to a smartphone, tablet, computer, or other connected devices where it can be viewed, analyzed, stored, or otherwise accessed by the user or other authorized party.

The blood pressure measurement device can be secured to a user in a variety of ways. For example, the blood pressure measurement device can removably couple to a variety of different straps, which can be configured to attach to different body parts of the user. In other cases, the blood pressure measurement device could be integrated into clothing, belts, hats, or other items worn by a user. In some cases, the blood pressure measurement device could be placed on a surface and the user could check their blood pressure by pressing a portion of their body against the device to measure their blood pressure.

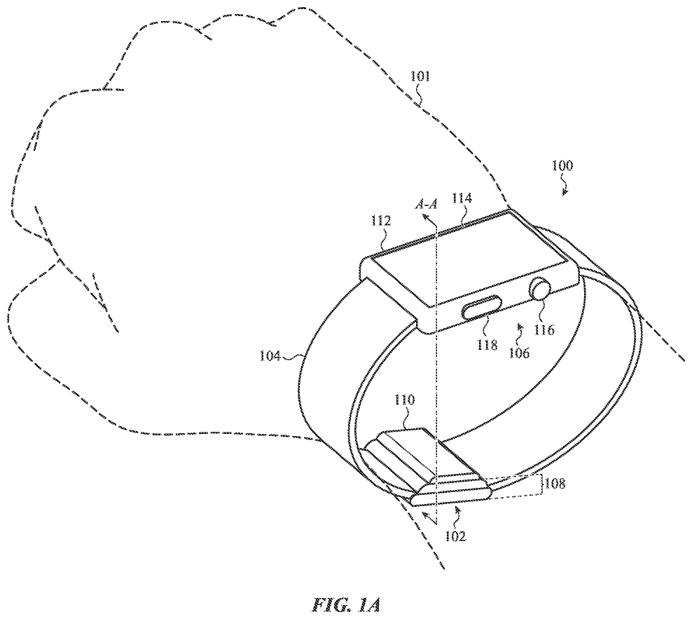

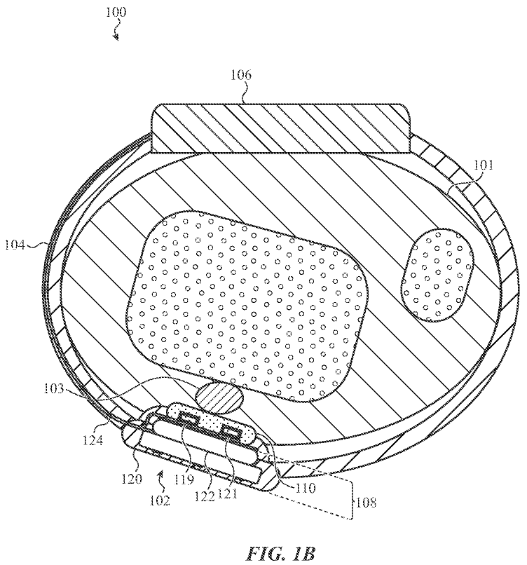

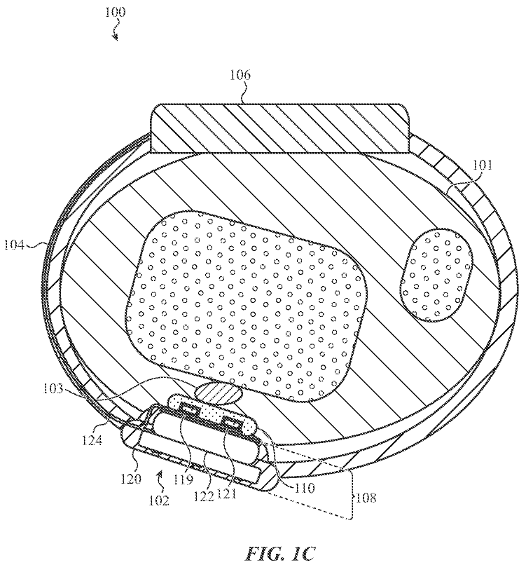

These and other embodiments are discussed below with reference to FIGS. 1-7. However, those skilled in the art will readily appreciate that the detailed description given herein with respect to these Figures is for explanatory purposes only and should not be construed as limiting.

FIG. 1A shows an example of a blood pressure measurement device 100 being worn on a limb of a user 101. The blood pressure measurement device 100 can include a pressure sensing stack 102, a strap 104 that secures the sensing stack 102 to the user 101, and an electronic device 106. The blood pressure measurement device 100 can worn around a limb of the user 101 such as around the wrist, upper arm or other portions of the user's arm, around a user's leg, or any other suitable location for measuring a blood pressure of the user 101. When worn by the user 101, the pressure sensing stack 102 can be operated to measure the user's blood pressure.

In some cases, the pressure sensing stack 102 can include an expansion mechanism 108 and a sensing mechanism 110. The expansion mechanism 108 can press the sensing mechanism 110 against a skin surface of a user 101 in the region of a blood vessel to begin collapsing the blood vessel. The sensing mechanism 110 can include one or more sensors (e.g., a vibration sensor, pressure sensor, and so on) that measure physiological parameters of the user as the sensing mechanism is pressed against the user 101. The expansion mechanism 108 can continue to press the sensing mechanism 110 against the user 101 until the blood vessel has become substantially collapsed and blood flow through the blood vessel has been stopped. In some cases, outputs from one or more sensors of the sensing mechanism 110 can be used to determine collapse of a blood vessel. The expansion mechanism 108, can then release the pressure applied to the user 101 until the blood vessel is no longer collapsed. Sensor measurements taken during the expansion and release of the expansion mechanism can be used to determine one or more blood pressure parameters of the user such as a diastolic blood pressure, a systolic blood pressure, a mean arterial pressure, or combination thereof. In some cases, the blood pressure measurements can be used to determine other blood pressure parameters such as tracking a pulse pressure wave, identifying one or more cardiac conditions such as abnormal heart rhythms, valve defects such as fibrillation, or the like.

In some cases, the strap 104 can be configured to apply a compressive force to the limb of the user to collapse a blood vessel of the user 101 during a blood pressure measurement. For example, the strap 104 can be configured to tighten around the limb to press the sensing mechanism 110 into the skin surface of a user. Pressure from the strap 104 can be in addition to the expansion mechanism 108 or as an alternative to the expansion mechanism 108.

In some cases, the strap 104 can be a passive structure that secures the pressure sensing stack 102 to the user 101. The strap 104 can be formed from polymer materials such as rubbers, silicone materials, thermoset materials, thermoplastic materials, fabrics, metals, ceramics, or any other suitable material or combination of different materials. In some embodiments, the pressure sensing stack 102 can include components such as a battery, a processor, and a wireless communication module that are used to gather and transmit data related to blood pressure measurements taken by the pressure sensing mechanism 110. In these cases, the blood pressure measurement device 100 may not include the electronic device 106, and instead include the pressure sensing stack 102 and the strap 104.

In some cases, the strap 104 can include one or more functional components used by the blood pressure sensing stack 102. For example, the strap 104 can house or couple to a battery, a processor, a wireless communication module, or the like. In these cases, the blood pressure measurement device 100 may not include the separate electronic device 106, and instead include the pressure sensing stack 102 and strap 104.

In other cases, the blood pressure measurement device 100 can include the electronic device 106. The electronic device 106 can be coupled to the user by the strap 104. The electronic device 106 can include a housing 112 and a display 114 that contain components such as a battery, processor(s) and memory, and wireless communication modules that are used to operate the pressure sensing stack 102 and analyze and display blood pressure measurement data for the user 101. In some embodiments, the strap 104 can include one or more electrical connections that electrically couple the pressure sensing stack 102 to the electronic device 106. In some embodiments, the electronic device 106 can be a wearable smart device such as a smartwatch.

The display 114 can be positioned under the cover and at least partially within the housing 112. The display 114 may define an output region in which graphical outputs are displayed. Graphical outputs may include graphical user interfaces, user interface elements (e.g., buttons, sliders, etc.), text, lists, photographs, videos, or the like. The display 114 may include a liquid-crystal display (LCD), organic light emitting diode display (OLED), or any other suitable components or display technology. In some cases, the display 114 may output a graphical user interface with one or more graphical objects that display information collected from or derived from the pressure-sensing system. For example, the display 114 may output one or more blood pressure measurement parameters to the user 101.

The display 114 may include or be associated with touch sensors and/or force sensors that extend along the output region of the display and which may use any suitable sensing elements and/or sensing techniques. Using touch sensors, the electronic device 106 may detect touch inputs applied to the cover, including detecting locations of touch inputs, motions of touch inputs (e.g., the speed, direction, or other parameters a gesture applied to the cover can generate), or the like. Using force sensors, the electronic device 106 may detect amounts or magnitudes of force associated with touch events applied to the cover. The touch and/or force sensors may detect various types of user inputs to control or modify the operation of the electronic device 106, including taps, swipes, multiple finger inputs, single- or multiple-finger touch gestures, presses, and the like.

The electronic device 106 may also include one or more user inputs such as a first input 116 having a cap, crown, protruding portion, or component(s) or feature(s) (collectively referred to herein as a “body”) positioned along a side surface of the housing 112. At least a portion of the first input 116 (such as the body) may protrude from, or otherwise be located outside, the housing 112, and may define a generally circular shape or circular exterior surface. The exterior surface of the body of the first input 116 may be textured, knurled, grooved, or otherwise have features that may improve the tactile feel of the first input 116 and/or facilitate rotation sensing.

The first input 116 may facilitate a variety of potential interactions. For example, the first input 116 may be rotated by a user (e.g., the crown may receive rotational inputs). Rotational inputs of the first input 116 may zoom, scroll, rotate, or otherwise manipulate a user interface or other object displayed on the display 114 among other possible functions. The first input 116 may also be translated or pressed (e.g., axially) by the user. Translational or axial inputs may select highlighted objects or icons, cause a user interface to return to a previous menu or display, or activate or deactivate functions among other possible functions. In some cases, the electronic device 106 may sense touch inputs or gestures applied to the first input 116, such as a finger sliding along the body of the first input 116 (which may occur when first input 116 is configured to not rotate) or a finger touching the body of the first input 116. In such cases, sliding gestures may cause operations similar to the rotational inputs, and touches on a cap or crown may cause operations similar to the translational inputs. As used herein, rotational inputs include both rotational movements of the first input 116, as well as sliding inputs that are produced when a user slides a finger or object along the surface of a crown in a manner that resembles a rotation (e.g., where the crown is fixed and/or does not freely rotate). In some embodiments, rotating, translating, or otherwise moving the first input 116 initiates a blood pressure measurement by a pressure sensing stack 102. In some cases, selecting an activity, requesting a location, specific movements of the user, and so on may also initiate pressure measurements by the pressure sensing stack 102.

The electronic device 106 may also include other inputs, switches, buttons, or the like. For example, the electronic device 106 includes a button 118. The button 118 may be a movable button or a touch-sensitive region of the housing 112. The button 118 may control various aspects of the electronic device 106. For example, the button 118 may be used to select icons, items, or other objects displayed on the display 114, to activate or deactivate functions (e.g., to silence an alarm or alert), or the like.

FIG. 1B shows an example cross-sectional view taken along section A-A shown in FIG. 1A of a blood pressure measurement device 100 being worn by a user 101. The blood pressure measurement device 100 can be operated to measure the blood pressure in a blood vessel 103 of a user 101. The sensing mechanism 110 can include a liquid filled chamber and a vibration sensor 119 can be placed in the liquid filled chamber. The vibration sensor can be configured to measure vibrations of blood flow within the blood vessel 103. In some cases, the sensing mechanism 110 can include a pressure sensor 121 that is configured to measure pressure in the blood vessel 103. The sensing stack 102 can be configured such that the sensing mechanism 110 is positioned between the expansion mechanism 108 and the user 101.

The expansion mechanism 108 can be configured to press the sensing mechanism 110 against the user 101 to collapse the blood vessel 103 (as shown in FIG. 1C). In some cases, the expansion mechanism 108 can include a pump 120 and an inflatable chamber 122 that is fluidly coupled to the pump 120. In other cases, the expansion mechanism 108 may include a closed expansion system, where the pump may include a reservoir which contains a volume of fluid and the pump can 120 can transfer fluid from the reservoir and into the inflatable chamber 122.

As used herein the term “fluidly coupled” is used to describe components that can exchange air using one or more sealed passages. The pump 120 can be attached to the strap 104 and can form a rigid structure such that as the inflatable chamber 122 is filled with air, it primarily expands towards the user. Accordingly, expansion of the inflatable chamber 122 pushes the sensing mechanism 110 against the user 101 to collapse the blood vessel 103. In this regard, the strap 104 and pump 120 can be configured to have sufficient rigidity such that as the inflatable chamber 122 expands, the primary motion of this expansion is toward the user 101.

In some cases, the pump 120 can include a rigid structure that reduces deformation or movement of the pump 120 during a measurement (e.g., when the inflatable chamber 122 is expanded). In some cases, the inflatable chamber 122 includes one or more compliant materials that allow the inflatable chamber 122 to expand. The compliant materials may be coupled to the pump 120 in any suitable manner including using adhesives, molding, coupling structures and so on.

The expansion mechanism 108 can be implemented in various ways. In some cases, the expansion mechanism 108 can be configured to tighten the strap 104 around the user 101 thereby compressing the pressure sensing mechanism 110 against the skin of the user 101 to collapse the blood vessel. In these cases, the expansion mechanism 108 can tighten the strap 104 using a ratcheting mechanism, a screw-based mechanism, gears, friction, a magnetically actuating element, a piezoelectric element, or other suitable system that tightens the strap around the user 101. In other cases, the expansion mechanism 108 can expand toward the user 101 using a piezoelectric actuator, pneumatic actuator, an electromechanical actuator, or combination thereof.

The sensing mechanism 110 can include a sealed chamber that contains a volume of liquid and the vibration sensor 119 can be configured to detect vibrations due to blood flow that are transferred from one or more blood vessels (e.g., blood vessels 103) and to the liquid in the sealed chamber. The pressure sensor 121 can be configured to detect pressure changes of the liquid volume.

The vibration sensor 119 and the pressure sensor 121 can output signals that are routed via one or more electrical connections 124, which can include one or more wires, cables, or other suitable structure. In some cases, the electrical connection 124 can carry other signals such as control signals to and from the expansion mechanism 108, signals for other sensors located on the pressure sensing stack 102, or any other electrical communication that occurs between the pressure sensing stack 102 and one or more external components such as the electronic device 106 or components coupled to the strap 104. In some cases, the pressure sensing stack 102 can include components that wirelessly transmit signals to a watch or other electronic device. These components may include memory, wireless antenna, a processor and other hardware and software used to transmit the measured sensor signals to a device using any suitable protocol including near field communications (NFC), Bluetooth, Wi-Fi and so on. These components may be part of the pressure sensing stack 102 and/or integrated into the strap 104.

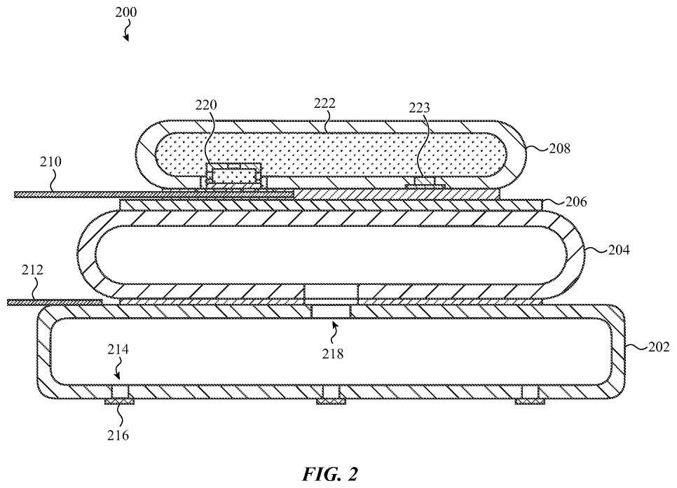

FIG. 2 shows a cross-sectional view of an example sensing stack 200. The sensing stack 200 can include an air pump 202, which can be an example of the air pumps described herein such as air pump 120; an inflatable chamber 204, which can be an example of the inflatable chambers described herein such as inflatable chamber 122; and a sensing chamber 208, which can be an example of the pressure sensing mechanism described herein such as sensing mechanism 110. The sensing stack 200 can include a support plate 206 positioned between the inflatable chamber 204 and the sensing chamber 208. The sensing stack 200 can also include a vibration sensor 220 and a first electrical connector 210 coupled to the vibration sensor 220, and a second electrical connector 212 coupled to the air pump 202.

The air pump 202 can be a piezoelectric air pump, diaphragm air pump, bellow air pump, reciprocating air pump, vane pump, or other suitable pump, or combination thereof. The air pump 202 can have a rigid housing that is attached to a strap of another structure used to secure the sensing stack 200 against a user. In this regard, the air pump 202 can form a rigid base structure that directs expansion of the inflatable chamber 204 toward the user.

In some cases, the air pump 202 can define one or more openings 214 for exchanging air with the external environment, one of which is labeled for simplicity. The openings 214 can be located on a first surface of the air pump 202, which may be referred to as a bottom surface. The air pump 202 can also include one or more membranes 216 that are coupled to the openings 214. The membrane(s) 216 can be configured to prevent water from entering the opening while allowing air to pass into and out of the air pump 202. In some cases, the membrane(s) 216 can be formed from a polytetrafluoroethylene (PTFE) material such as an expanded (ePTFE) material, or other suitable material.

The inflatable chamber 204 can be positioned on a second surface of the air pump 202, which may be referred to as a top surface. The inflatable chamber can be formed from an expandable material such as rubber, silicone elastomer, or polymer compound that can expand at air pressures that are used to collapse a blood vessel of a user. In some cases, a bottom surface of the inflatable chamber 204 can be coupled to the top surface of the air pump 202. The inflatable chamber 204 can be coupled to the air pump 202 in a variety of ways including the use of adhesives, welding, laminating, molding and/or overmolding, using mechanical fasteners or other suitable technique, or combinations thereof. The sensor stack 200 can be assembled to define an air passage 218 between the air pump 202 and the inflatable chamber 204. In some cases, the air passage 218 can be on opening, and in other cases the air passage 218 can include a valve, membrane or other structure that controls the direction of air flow between the air pump 202 and the inflatable chamber 204.

The positioning of the bottom surface of the inflatable chamber 204 on the top surface of the air pump 202, can direct the expansion of the inflatable chamber 204 toward a user. For example, a rigidity of the air pump 202 and a strap coupled to the air pump 202 can be configured such that the inflatable chamber 204 primarily compresses the tissue of the user to collapse the blood vessel with little movement of the air pump 202 away from a user. In some cases, flexible material can surround (or partially surround) the inflatable chamber 204, which may help protect or otherwise cover the inflatable chamber 204. In other cases, a rigid barrier may partially surround the inflatable chamber 204 (e.g., around a periphery of the inflatable chamber 204), which can help direct expansion of the inflatable chamber toward the user.

The support plate 206 can be configured to help distribute the force of the inflatable chamber 204 across the sensing chamber 208 as the inflatable chamber 204 is expanded. Additionally or alternatively, the support plate 206 can prevent blood pressure pulse signal loss between the pressure sensing chamber and the inflatable chamber. The support plate 206 can be formed from a rigid or semi-rigid material that has low deformation at the pressures that are used to compress a blood vessel. For example, the support plate 206 can be formed from a metal, plastic, ceramic, or other suitable material. In some cases, the support plate 206 can be sized to distribute a force created by the inflatable chamber 204 across a bottom surface of the sensing chamber 208. For example, a profile of the top surface of the support plate 206 can be sized to equal or encompass a profile of the bottom surface of the sensing chamber 208. The support plate 206 can be coupled to the inflatable chamber 204 and/or the sensing chamber 208 in a variety of ways including the use of adhesives, welding, molding and/or overmolding, using mechanical fasteners, or combinations thereof.

The sensing chamber 208 can include a flexible outer housing that contains a liquid 222. The sensing chamber 208 can include a vibration sensor 220 that is configured to detect vibrations of the liquid 222. In some cases, the vibration sensor 220 contains liquid that is fluidly connected with the liquid 222 in the sensing chamber 208 such that fluid can move between these components to measure vibrations inside the sensing chamber 208. In some cases, the vibration sensor 220 can be completely or partially contained within the sensing chamber 208. In either case, the first electrical connector 210 can be configured to communicably couple the vibration sensor 220 to one or more other components of the system such as a processor. In some cases, the electrical connector 210 can be a flex circuit.

The liquid 222 can be any suitable liquid such as water or water-based liquids, oil-based liquids such as a hydraulic liquid, or combinations thereof. The sensing chamber 208 can have a liquid plug 223 to fill liquid and seal the sensing chamber 208. In some cases, the plug and/or filling process can be configured to prohibit or remove any air bubbles or gaseous voids inside the sensing chamber 208. In some embodiments, the sensing chamber 208 can be completely filled with the liquid, for example, such that there are no gas/air bubbles within the sensing chamber. The sensing chamber 208 can be sealed so that the volume liquid remains constant during a measurement process.

The vibration sensor 220 can include accelerometers, piezoelectric sensors, a microphone, microelectromechanical (MEMS) sensor and/or any other suitable vibration sensing structures that are configured to output an electrical signal that is indicative of vibrations in the liquid 222. The vibration sensor 220 can also include a cover that defines one or more openings. The cover can surround one or more portions of the vibration sensor 220 and can be configured to help prevent air bubbles from forming around the vibration sensor. In some cases, the vibration sensor 220 (e.g., cover) can include one or more ports or openings, which may help fill the sensor with liquid.

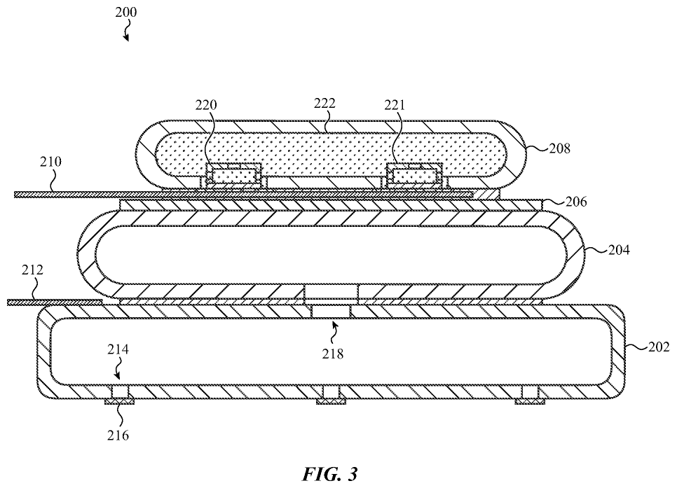

FIG. 3 shows a cross-sectional view of another example of the sensing stack 200. In the example shown in FIG. 3, the sensing stack 200 can include a pressure sensor 221. The pressure sensor 221 can include a pressure transducer that is configured to output an electrical signal that is indicative of a pressure of the liquid 222. The pressure sensor 221 can also include a cover that defines one or more openings. The cover can surround the pressure sensor and can be configured to help prevent air bubbles from forming around the pressure sensor.

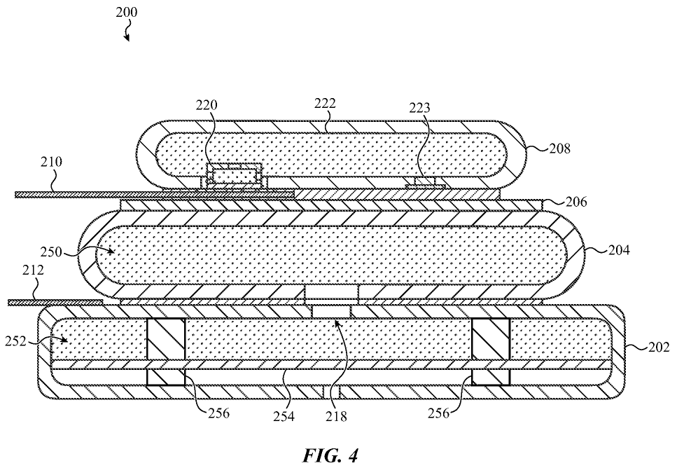

FIG. 4 shows a cross-sectional view of another example of the sensing stack 200 that includes a closed expansion system. The closed expansion system can be implemented in a variety of ways. Generally and broadly, the closed expansion system can include a reservoir that contains a fluid that can be used to expand and contract the inflatable chamber 204 by moving fluid to or from the reservoir and into the inflatable chamber 204. For example, the inflatable chamber 204 that contains a first portion of fluid 250 and the pump 202 may define a second internal chamber that contains a second portion of fluid 252 (e.g., a reservoir). The first and second chambers can be fluidly coupled and the pump 202 may include a mechanism that moves fluid between the fluid between the chambers.

In some embodiments, the pump 202 can include a piston 254 that moves in a first direction to move fluid into the inflatable chamber 204 to increase a volume of the first fluid portion 250 (while decreasing the volume of the second fluid portion 252) to expand the inflatable chamber 204 as described herein. The piston 254 may move in a second direction (e.g., opposite the first direction) to move fluid from the inflatable chamber 204 and into the pump 202. The piston 254 may be driven by one or more linear actuators 256. In other cases, closed system can included an air pump that generates compressed/higher pressure air to drive the piston 254. In these cases, the piston 254 may be configured to generate an increased hydraulic pressure in the inflatable chamber. Other suitable techniques may be used to move the fluid between the reservoir and the inflatable chamber 204. For example, an suitable fluid pump could be used to move fluid between the first fluid portion 252 and the second fluid portion 254. In other cases, the fluid reservoir can be separate from the pump 202, for example, the fluid reservoir containing the second fluid portion 252 could be located in a band of a watch or on another portion of the sensing stack. The closed expansion system may therefore contain the fluid that is used to expand and contract the inflatable chamber 204, for example, as opposed to using air (or liquid) from the surrounding environment.

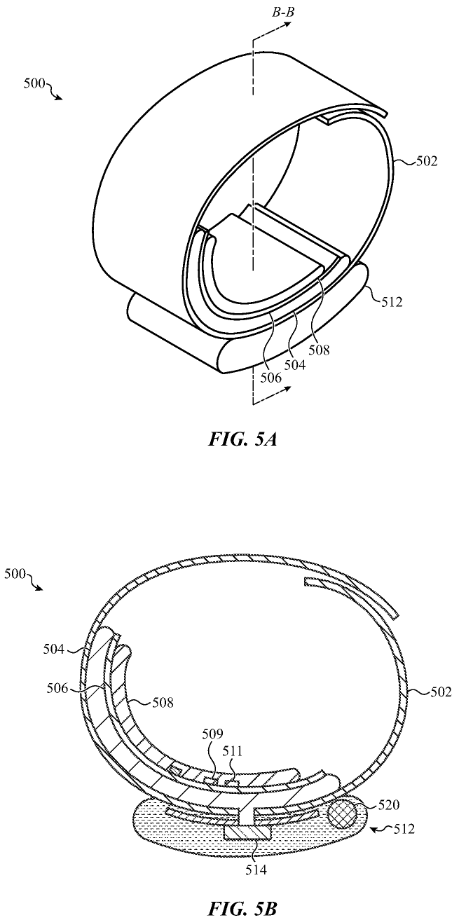

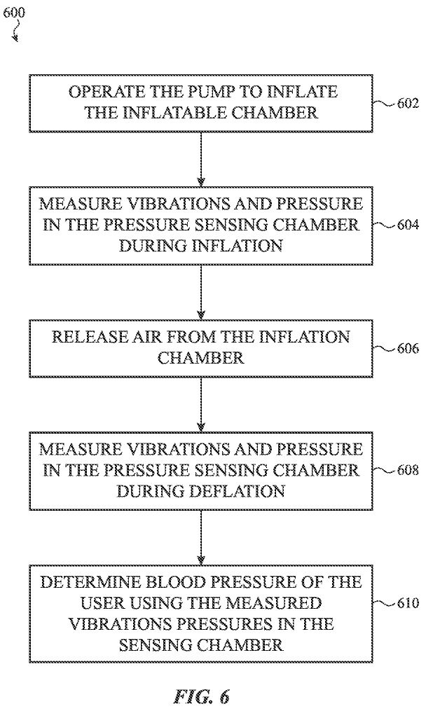

FIG. 5A shows a perspective view of an example blood pressure measurement device 500. The blood pressure measurement device 500 can include any or all of: a strap 502, which may be any of the straps described herein; an inflatable chamber 504, which may be any of the inflatable chambers described herein; a support plate 506, which may be any of the support plates described herein; and a sensing chamber 508, which may be any of the sensing chambers described herein. The blood pressure measurement device 500 can also incorporate any or all of: a control system 512 including an pump 514 (shown in FIG. 5B), which may be any of the pumps described herein; an integrated chip (not shown) configured to control operation of the blood pressure measurement device 500; and a battery 520 (shown in FIG. 5B) or other power source.

FIG. 5B shows a cross-sectional view taken along line B-B of the example blood pressure measurement device 500 shown in FIG. 5A. A vibration sensor 509 can be positioned in the sensing chamber 508. In some embodiments, a pressure sensor 511 can also be positioned in the sensing chamber 508.

The inflatable chamber 504, the support plate 506 and the sensing chamber 508 extend along the strap 502 such that, when the device is worn by a user, these components substantially encircle a portion or an entirety of the user's limb. The strap 502 can be coupled together at opposite ends to secure the blood pressure measurement device 500 to the user. In these examples, the sensing chamber 508 may extend over a larger area of a user's limb, which provides a greater surface area for transferring vibrations and/or pressure pulses from one or more blood vessels to the liquid in the sensing chamber. This may increase a sensitivity and/or accuracy of blood pressure measurements taken by the blood pressure measurement device 500.

In some cases, having the sensing chamber 508 substantially encircle the limb of a user can allow the device to be positioned in a variety of orientations with respect to the user's limb while performing blood pressure measurements on the user. For example, the blood pressure measurement device 500 can be positioned at different angular orientations around a body part such as by rotating the device while it is being worn by the user. Embodiments of the blood pressure measurement device 500 can be configured to wrap around and/or contact various body parts of a user such as a head, torso, leg, bicep, and so on.

In some embodiments, the support plate 506 can bend around the limb of a user while still being sufficiently rigid to distribute forces along the sensing chamber 508 that are generated by the inflatable chamber 504. For example, the support plate 506 can be formed from a thin and rigid material such as a thin metal sheet (e.g., spring steel, nitinol, or the like), plastic or plastic composites such as a woven carbon fiber material, or other suitable material or structure. In some cases, the support plate 506 is formed from multiple segments such as a band with multiple discrete links that allow the support plate 506 to conform around a body part. The segments forming the support plate can be made from rigid or semi-rigid materials such as metals, plastics, or other suitable materials.

In some cases, the sensing chamber 508, support plate 506, and the inflatable chamber 504 may be positioned on the blood pressure measurement device 500 such that, when the device is worn by a user, these components are positioned proximate to one or more of the user's blood vessels. For example, the blood pressure measurement device 500 can include features that help position the sensing chamber 508 in a specific orientation relative to a user's limb. For example, the blood pressure measurement device may be rotated around a limb or other body part of a user to orient the sensing chamber 508 relative to a blood vessel or other bodily feature. These can include features that interface with a user's anatomy, features that direct a user to wear the device in a specific orientation, and so on. In some cases, the blood pressure measurement device 500 can instruct the user to position or adjust the position of the device to achieve a desired orientation relative to a limb, such as by providing audio instructions, visual instructions such as using a display output, or a combination thereof. In some cases, measurement data from the sensing chamber 508 or other sensors may be used to help direct positioning of the device relative to a limb of a user.

FIG. 6 shows an example process 600 for operating a blood pressure measurement device such as the blood pressure measurement devices described herein.

At 602, the process 600 can include operating the pump to inflate the inflatable chamber. In some cases, the pump can inflate the chamber to a defined pressure that is above the pressure that causes collapse of a user's blood vessel. In other cases, vibration measurements and/or pressure measurements can be used to control the maximum inflation pressure.

At 604, the process 600 can include measuring vibrations in the sensing chamber during inflation of the inflatable chamber. As the inflatable chamber presses the sensing chamber against the user, the vibrations sensor may detect vibrations from blood flowing through one or more blood vessels. In some cases, the increased pressure of the sensing chamber against the user may increase a vibration coupling between the sensing chamber and the user, which increases a sensitivity, intensity and or quality of the vibration measurements. In some cases, measuring vibrations may include measured blood flow sounds that would be observed as part of an auscultatory measurement process. The blood flow measurement system can record and analyze the blood flow sounds to determine one or more blood flow parameters as described herein.

In some embodiments, the blood pressure measurements device may include an external vibration sensor that is positioned outside the sensing chamber. For example, the external vibration sensor can be positioned on the pump and/or incorporated into a companion electronic device such as a smart watch. The external vibration sensor may be sufficiently separated from the user and operate to remove noise (e.g., due to the pump, movement of the user, environmental) from the vibration measurements made by the vibration sensor positioned in the sensing chamber. For example, the external vibration sensor may be positioned to detect vibrations due to operation of an pump (or other components of the system), while being isolated from the user so as to minimize the detection of blood flow vibrations. In these cases, measurements from the external vibration sensor can be used to remove noise from the vibration sensor positioned in the sensing chamber.

The vibration measurements can be used by the blood pressure measurement device to identify physiological parameters of a user including Korotkoff sounds, heart rates, pulse pressure profiles, opening and/or closing of a heart valve and so on. For example, outputs from the vibration sensor may be used to determine partial and/or complete collapse of a blood vessel, which may be used to determine blood pressure parameters of a user such as a diastolic pressure, systolic pressure, and so on.

At 604, the process 600 can also include measuring pressure in the sensing chamber during inflation of the inflatable chamber. As the pump is operating to inflate the inflatable chamber, the pressure sensing device can be operated to measure blood pressures of a user. In some cases, pressure signal outputs from the pressure sensing device can be used to control an inflation rate, maximum inflation pressure, or both. The pressure measurements can be correlated to the vibration measurements, which can be used to associate pressure measurements with physiological events such as partial or complete collapse of a blood vessel.

At 606, the process 600 can include releasing the air from the inflation chamber. In some cases, the air release can be controlled by one or more valves coupled to the inflatable chamber. The release rate of air can be controlled via the valve(s). In some cases, pressure signal outputs from the pressure sensing device can be used to control a deflation rate.

At 608, the process 600 can include measuring vibrations and/or pressure in the sensing chamber during deflation of the inflatable chamber. For example, as the inflatable chamber is being deflated, the vibration sensor can be operated to measure vibrations due to changes in blood flow as the vessel transitions from a collapsed state to an open state. Additionally, the pressure sensing device can be operated to measure blood pressures as the vessel transitions from a collapsed state to an open state.

At 610, the process 600 can include determining a blood pressure of a user using the measured vibrations and pressures during inflation, deflation or a combination thereof. The blood pressure measurements taken during the expansion and release of the expansion mechanism can be used to determine one or more blood pressure parameters of a user such as a diastolic blood pressure, a systolic blood pressure, a mean arterial pressure, or combination thereof. In some cases, the blood pressure measurements can be used to determine other blood pressure parameters such as tracking a pulse pressure wave, identifying one or more cardiac conditions such as abnormal heart rhythms, valve defects such as fibrillation, or the like.

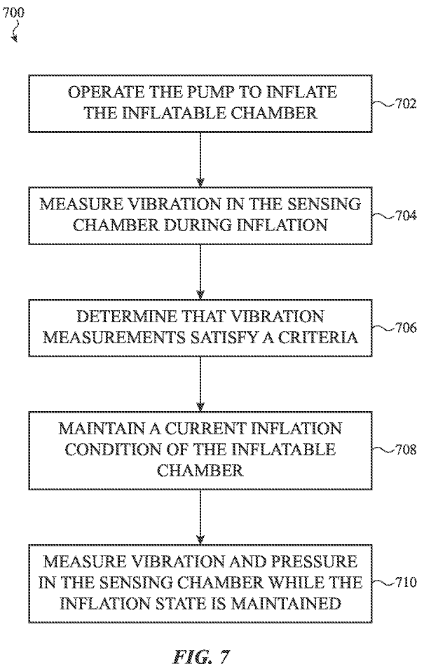

FIG. 7 shows an example process 700 for operating a blood pressure measurement device such as the blood pressure measurement devices described herein.

At 702, the process 700 can include operating the pump to inflate the inflatable chamber, as described herein. In some cases, the inflatable chamber can be inflated at a constant rate. In other cases, the inflatable chamber may be inflated according to an inflation profile. For example, an inflation profile could include a step profile that inflates the chamber in one or more increments. In other cases, the inflatable chamber may inflate based on outputs from the vibration sensor, pressure sensor and/or other sensors.

At operation 704, the process 700 can include measuring vibrations in the sensing chamber during inflation, as described herein. Outputs from the vibration sensor may be used to identify blood flow parameters of the user, such as a pulse profile, partial or complete collapse of a blood vessel, heart valve sounds and/or the like. For example, as the inflatable chamber is expanded and the sensing chamber is pressed against the user to collapse a blood vessel vibrations from the blood flow may be transferred to the fluid in the sensing chamber. Pressing the sensing chamber against the user may increase an acoustic coupling between the blood flow and the liquid in the sensing chamber. Accordingly, the vibration sensor may measure changes in vibration that result from blood flow such as acceleration of blood velocity during systole and deceleration of blood velocity during diastole. The vibration measurement may be analyzed to identify parameters such as systole timing, diastole timing, heart rate, variations in beat-to-beat timing and so on. In some cases, the vibration measurements may be analyzed to identify heart valve sounds, which may indicate functioning of a heart valve such as whether incomplete valve opening or closure or other conditions. Analysis of the vibration measurements may include use of machine learning algorithms and/or any other suitable qualitative and/or quantitative data analysis techniques.

At operation 706, the process 700 can include determining if the vibration measurements satisfy a criteria. In some cases, the criteria can include identifying a maximum vibration condition during the inflation process. For example, as the inflatable chamber is inflated and the pressure on the blood vessel is increased, the vibration sensor may measure blood flow vibrations as described herein. The inflatable chamber may be inflated until the vessel is completely collapsed (or at some other defined point, such as a defined pressure in the liquid chamber). The vessel collapse may be identified using the vibration measurements and/or pressure measurements. The blood pressure measurement device can analyze the vibration data to determine when during the inflation process the measured vibrations were greatest. In other cases, the blood pressure measurement device may be configured with a defined vibration threshold and the device can determine at which point in the inflation process the vibration outputs meet the defined vibration threshold. In some cases, the blood pressure measurement device may use the identified vibration condition (e.g., maximum vibration output condition) to determine an inflation state of the inflatable chamber. The inflation state may be the pressure of the liquid in the sensing chamber as measured by the pressure sensor, an inflation volume of the inflatable chamber, an inflation time from the start of the inflation process, a determination from the vibration measurements and so on. In some cases, the criteria may be determined using a deflation procedure as opposed to an inflation procedure or one or more inflation and deflation procedures, a partial inflation procedure (e.g., inflating the inflation chamber until the measured vibration signal satisfies a defined threshold or value), a partial deflation procedure (e.g., deflating the inflation chamber until the measured vibration signal satisfies a defined threshold or value), and so on.

At operation 708, the process 700 can include maintaining a current inflation condition of the inflatable chamber. For example, in response to identifying a maximum sound output, the blood pressure measurement device may determine a pressure of the liquid in the sensing chamber at the maximum sound output condition and inflate (or deflate) the inflation chamber until the identified pressure is reached. The blood pressure measurement device may maintain the identified pressure in the inflation chamber.

At operation 710, the process 700 can include measuring vibrations and/or pressure in the sensing chamber while the inflation state is maintained. The blood pressure measurement device may measure vibrations for a defined period, until a user input is received or until a second criteria is satisfied (e.g., for a defined number of heart beats).

In some cases, the blood flow measurement device may be able to measure blood flow for a period of time at various different compression states of a vessel. For example, the blood flow measurement device may perform a step sweep by inflating the chamber to a first defined inflation condition (e.g., first pressure in the liquid sensing chamber), measure vibrations at that first defined inflation condition for a first period of time, and inflate to a second defined inflation condition (e.g., second pressure in the liquid sensing chamber) and measure vibrations at the second defined inflation condition for a second period of time, and so on.

The blood pressure measurement device may determine one or more physiological parameters by measuring vibrations at a particular inflation condition. For example, pressure on the vessel may increase acoustic coupling between the blood vessel and the sensing chamber, which may help measure blood flow sounds and determine physiological parameters such as a blood flow profile over one or more heart beats, heart rates, variations in heart rate, valve conditions such as closure of one or more or the heart valves and so on.

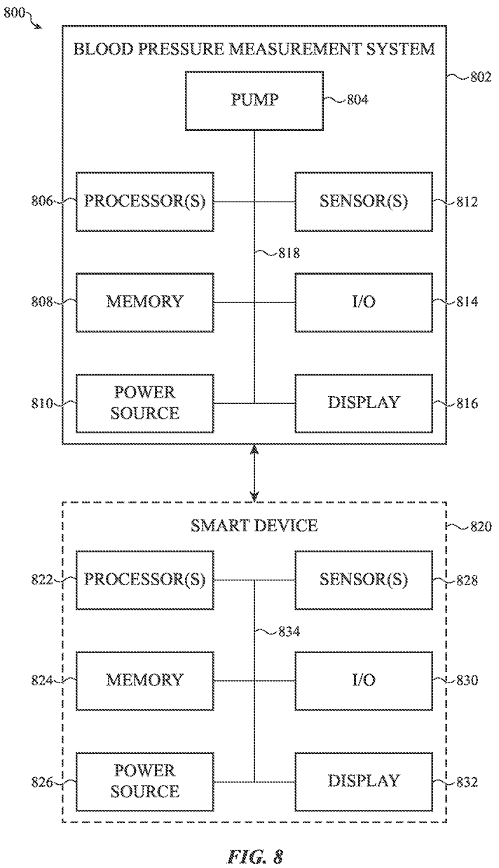

FIG. 8 shows an example system diagram for a blood pressure measurement system 800, which may in some cases take the form of any of the blood pressure measurement devices or components thereof described with reference to FIGS. 1-7. In some cases, the blood pressure measurement system 800 can include a blood pressure measurement device 802 that is configured to detect one or more blood pressure parameters of a user. For example, the blood pressure measurement device 802 can include components that analyze sensor outputs and display measurement data to a user. In some embodiments, the blood pressure measurement device 802 can interface with a smart device 820 such as a smartwatch, a smartphone, tablet, wearable device such as a health monitoring device, and so on. In these cases, the blood pressure measurement device 802 may utilize components from the smart device 820 for performing blood pressure measurements. For example, the blood pressure measurement device 802 can send outputs from its sensors such as a pressure sensor to the smart device 820, and the smart device 820 can analyze, and display the data to a user.

The blood pressure measurement device 802 can include an pump 804, a processor 806, memory 808, a power source 810, one or more sensors 812, an input/output (I/O) mechanism 814, and a display 816.

The pump 804 can be an example of the pumps described herein and be operable to inflate the inflatable bladder to pressures that collapse a blood vessel of a user. The pump 804 can be an ultrasonic pump, diaphragm pump, bellow pump, reciprocating pump, vane pump, or other suitable pump, or combination thereof.

The processor 806 can control some or all of the operations of the blood pressure measurement device 802. The processor 806 can communicate, either directly or indirectly, with some or all of the components of the blood pressure measurement device 802. For example, a system bus or other communication mechanism 818 can provide communication between the pump 804, processor 806, the memory 808, the power source 810, the sensor(s) 812, the input/output (I/O) mechanism 814, and the display 816.

The processor 806 can be implemented as any electronic device capable of processing, receiving, or transmitting data or instructions. For example, the processor 806 can be a microprocessor, a central processing unit (CPU), an application-specific integrated circuit (ASIC), a digital signal processor (DSP), or combinations of such devices. As described herein, the term “processor” is meant to encompass a single processor or processing unit, multiple processors, multiple processing units, or other suitable computing element or elements.

It should be noted that the components of the blood pressure measurement device 802 can be controlled by multiple processors. For example, select components of the blood pressure measurement device 802 (e.g., a sensor 812) may be controlled by a first processor and other components of the blood pressure measurement device 802 (e.g., the I/O 814) may be controlled by a second processor, where the first and second processors may or may not be in communication with each other.

The memory 808 can store electronic data that can be used by the blood pressure measurement device 802. For example, the memory 808 can store electrical data or content such as, for example, audio and video files, documents and applications, device settings and user preferences, timing signals, control signals, and data structures or databases. The memory 808 can be configured as any type of memory. By way of example only, the memory 808 can be implemented as random access memory, read-only memory, Flash memory, removable memory, other types of memory storage elements, or combinations of such devices.

The power source 810 can be implemented with any device capable of providing energy to the blood pressure measurement device 802. For example, the power source 810 may be one or more batteries or rechargeable batteries. Additionally or alternatively, the power source 810 can be a power connector or power cord that connects the blood pressure measurement device 802 to another power source, such as a wall outlet.

The blood pressure measurement device 802 may also include one or more sensor(s) 812 positioned almost anywhere on the blood pressure measurement device 802. The sensor(s) 812 can be configured to sense one or more type of parameters, such as but not limited to, pressure, sound, light, touch, heat, movement, relative motion, biometric data (e.g., biological parameters), and so on. For example, the sensor(s) 812 may include a pressure sensor, an auditory sensor, a heat sensor, a position sensor, a light or optical sensor, an accelerometer, a pressure transducer, a gyroscope, a magnetometer, a health monitoring sensor, and so on. Additionally, the one or more sensor(s) 812 can utilize any suitable sensing technology, including, but not limited to, capacitive, ultrasonic, resistive, optical, ultrasound, piezoelectric, and thermal sensing technology.

The I/O mechanism 814 can transmit and/or receive data from a user or another electronic device. An I/O mechanism 814 can include a display, a touch sensing input surface, one or more buttons (e.g., a graphical user interface “home” button), one or more cameras, one or more microphones or speakers, one or more ports, such as a microphone port, and/or a keyboard. Additionally or alternatively, an I/O device or port can transmit electronic signals via a communications network, such as a wireless and/or wired network connection. Examples of wireless and wired network connections include, but are not limited to, cellular, Wi-Fi, Bluetooth, IR, and Ethernet connections.

The blood pressure measurement device 802 may also include a display 816. The display 816 may include a liquid-crystal display (LCD), organic light-emitting diode (OLED) display, light-emitting diode (LED) display, or the like. If the display 816 is an LCD, the display 816 may also include a backlight component that can be controlled to provide variable levels of display brightness. If the display 816 is an OLED or LED type display, the brightness of the display 816 may be controlled by modifying the electrical signals that are provided to display elements. The display 816 may correspond to any of the displays shown or described herein.

The smart device 820 can include a processor 822, memory 824, a power source 826, one or more sensors 828, an input/output (I/O) mechanism 830, and a display 832.

The processor 822 can control some or all of the operations of the smart device 820. The processor 822 can communicate, either directly or indirectly, with some or all of the components of the smart device 820. For example, a system bus or other communication mechanism 834 can provide communication between the processor 822, the memory 824, the power source 826, the sensor(s) 828, the input/output (I/O) mechanism 830, and the display 832.

The processor 822 can be implemented as any electronic device capable of processing, receiving, or transmitting data or instructions. For example, the processor 822 can be a microprocessor, a central processing unit (CPU), an application-specific integrated circuit (ASIC), a digital signal processor (DSP), or combinations of such devices. As described herein, the term “processor” is meant to encompass a single processor or processing unit, multiple processors, multiple processing units, or other suitable computing element or elements.

It should be noted that the components of the smart device 820 can be controlled by multiple processors. For example, select components of the smart device 820 (e.g., a sensor 828) may be controlled by a first processor and other components of the smart device 820 (e.g., the I/O 830) may be controlled by a second processor, where the first and second processors may or may not be in communication with each other.

The memory 824 can store electronic data that can be used by the smart device 820. For example, the memory 824 can store electrical data or content such as, for example, audio and video files, documents and applications, device settings and user preferences, timing signals, control signals, and data structures or databases. The memory 824 can be configured as any type of memory. By way of example only, the memory 824 can be implemented as random access memory, read-only memory, Flash memory, removable memory, other types of storage elements, or combinations of such devices.

The power source 826 can be implemented with any device capable of providing energy to the smart device 820. For example, the power source 826 may be one or more batteries or rechargeable batteries. Additionally or alternatively, the power source 826 can be a power connector or power cord that connects the smart device 820 to another power source, such as a wall outlet.

The smart device 820 may also include one or more sensor(s) 828 positioned almost anywhere on the smart device 820. The sensor(s) 828 can be configured to sense one or more type of parameters, such as but not limited to, pressure, sound, light, touch, heat, movement, relative motion, biometric data (e.g., biological parameters), and so on. For example, the sensor(s) 828 may include a pressure sensor, an auditory sensor, a heat sensor, a position sensor, a light or optical sensor, an accelerometer, a pressure transducer, a gyroscope, a magnetometer, a health monitoring sensor, and so on. Additionally, the one or more sensor(s) 828 can utilize any suitable sensing technology, including, but not limited to, capacitive, ultrasonic, resistive, optical, ultrasound, piezoelectric, and thermal sensing technology.

The I/O mechanism 830 can transmit and/or receive data from a user or another electronic device. An I/O mechanism 830 can include a display, a touch sensing input surface, one or more buttons (e.g., a graphical user interface “home” button), one or more cameras, one or more microphones or speakers, one or more ports, such as a microphone port, and/or a keyboard. Additionally or alternatively, an I/O device or port can transmit electronic signals via a communications network, such as a wireless and/or wired network connection. Examples of wireless and wired network connections include, but are not limited to, cellular, Wi-Fi, Bluetooth, IR, and Ethernet connections.

The smart device 820 may also include a display 832. The display 832 may include a liquid crystal display (LCD), organic light-emitting diode (OLED) display, light-emitting diode (LED) display, or the like. If the display 832 is an LCD, the display 832 may also include a backlight component that can be controlled to provide variable levels of display brightness. If the display 832 is an OLED or LED type display, the brightness of the display 832 may be controlled by modifying the electrical signals that are provided to display elements. The display 832 may correspond to any of the displays shown or described herein.

As described above, one aspect of the present technology is determining physiological parameters of a user such as blood pressure metrics, and the like. The present disclosure contemplates that in some instances this gathered data may include personal information data that uniquely identifies or can be used to contact or locate a specific person. Such personal information data can include demographic data, location-based data, telephone numbers, email addresses, Twitter IDs (or other social media aliases or handles), home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to provide haptic or audiovisual outputs that are tailored to the user. Further, other uses for personal information data that benefit the user are also contemplated by the present disclosure. For instance, health and fitness data may be used to provide insights into a user's general wellness or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (“HIPAA”); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of determining spatial parameters, the present technology can be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data at a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, haptic outputs may be provided based on non-personal information data or a bare minimum amount of personal information, such as events or states at the device associated with a user, other non-personal information, or publicly available information.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A blood pressure measurement device, comprising:
a strap operable to couple the blood pressure measurement device to a user;
a pump coupled to the strap;
an inflatable chamber fluidly coupled to the pump and configured to expand towards the user when inflated;
a sensing chamber coupled to the inflatable chamber and configured to be compressed between the inflatable chamber and the user when the blood pressure measurement device is coupled to the user, the sensing chamber containing a liquid;
a pressure sensor positioned in the sensing chamber and configured to detect a pressure of the liquid; and
a vibration sensor positioned in the sensing chamber and configured to detect vibrations due to a blood flow of the user.

2. The blood pressure measurement device of claim 1, wherein:
the strap is coupled to a smartwatch and configured to:
transmit or route one or more first signals from the smartwatch to the pump, the one or more first signals controlling inflation and deflation of the pump;
transmit or route one or more second signals from the pressure sensor to the smartwatch, the one or more second signals indicative of the measured pressures within the sensing chamber;
transmit or route one or more third signals from the pressure sensor to the smartwatch, the one or more third signals indicative of measured vibrations due to the blood flow of the user; and
the smartwatch is operable to determine a blood pressure of the user using the one or more first signals, second signals, and third signals.

3. The blood pressure measurement device of claim 2, wherein:
the pressure sensor is a first pressure sensor;
the smartwatch further comprises a second pressure sensor operable to measure a pressure of an ambient environment; and
the smartwatch is configured to further determine the blood pressure of the user using the measured pressure of the ambient environment.

4. The blood pressure measurement device of claim 2, wherein:
the smartwatch further comprises an accelerometer operable to measure movement of the user; and
the smartwatch is configured to determine the blood pressure of the user using the measured movement of the user.

5. The blood pressure measurement device of claim 2, wherein:
the sensing chamber comprises a flexible outer wall that contains the liquid;
the flexible outer wall is configured to conform to a surface of the user when the blood pressure measurement device is worn by the user; and
a portion of the flexible outer wall is surrounded by a rigid barrier that prevents expansion of the flexible outer wall.

6. The blood pressure measurement device of claim 1, wherein the liquid comprises an oil.

7. The blood pressure measurement device of claim 1, further comprising a support plate positioned between the inflatable chamber and the sensing chamber.

8. The blood pressure measurement device of claim 1, wherein the pump is configured to be positionable along the strap.

9. A blood pressure measurement device, comprising:
a strap operable to couple the blood pressure measurement device to a user, the strap comprising an inflation chamber configured to expand toward the user when inflated;
a support plate coupled to the strap and positioned between the strap and the user when the blood pressure measurement device is worn by the user;
a sensing chamber coupled to the support plate, positioned between the support plate and the user when the blood pressure measurement device is coupled to the user, and containing a liquid;
a pressure sensor positioned in the sensing chamber and configured to detect a pressure of the liquid; and
a vibration sensor positioned in the sensing chamber and configured to detect vibrations due to blood flow of the user.

10. The blood pressure measurement device of claim 9, further comprising:
a pump fluidly coupled to the inflation chamber; and
a processor configured to control the pump to inflate and deflate the inflation chamber.

11. The blood pressure measurement device of claim 10, wherein the processor is configured to:
receive pressure measurements from the pressure sensor during inflation of the inflation chamber;
receive vibration measurements from the vibration sensor during the inflation of the inflation chamber; and
determine a blood pressure of the user using the pressure measurements and the vibration measurements.

12. The blood pressure measurement device of claim 10, wherein the processor is configured to:
receive pressure measurements from the pressure sensor during deflation of the bladder;
receive vibration measurements from the vibration sensor during deflation of the bladder; and
determine a blood pressure of the user using the pressure measurements and the vibration measurements.

13. The blood pressure measurement device of claim 10, wherein the processor is configured to:
receive vibration measurements during inflation of the bladder;
in response to the vibration measurements satisfying a criteria, maintain a current inflation condition of the bladder; and
measure pressure in the sensing chamber while maintaining the current inflation condition of the bladder.

14. The blood pressure measurement device of claim 10, further comprising a battery, wherein the battery and the processor are coupled to the strap.

15. The blood pressure measurement device of claim 10, wherein:
the vibration sensor is a first vibration sensor;
the blood pressure measurement device comprises a second vibration sensor positioned outside the sensor chamber; and
the processor is configured to determine a blood pressure of the user using outputs from the first vibration sensor and the second vibration sensor.

16. The blood pressure measurement device of claim 10, wherein:
the pressure sensor is a first pressure sensor;
the blood pressure measurement device comprises a second pressure sensor positioned outside the sensor chamber; and
the processor is configured to determine a blood pressure of the user using outputs from the first pressure sensor and the second pressure sensor.

17. A blood pressure measurement system, comprising:
a smartwatch comprising:
a housing;
a display positioned at least partially within the housing;
a processor positioned in the housing;
a blood pressure measurement device comprising:
an inflatable chamber configured to expand towards a user when inflated;
a sensing chamber coupled to the inflatable chamber and configured to be placed between the inflatable chamber and the user, the sensing chamber containing a liquid;
a pressure sensor positioned in the sensing chamber and configured to detect a pressure of the liquid; and
a vibration sensor positioned in the pressure chamber and configured to detect vibrations due to blood flow of the user; and
a strap configured to:
couple the smartwatch to a first location on the user;
couple the blood pressure measurement device to a second location on the user; and
transmit one or more signals between the smartwatch and the blood pressure measurement device.

18. The blood pressure measurement system of claim 17, wherein the processor is configured to:
control inflation and deflation of the inflatable chamber;
receive pressure measurements from the pressure sensor;
receive vibration measurements from the vibration sensor; and
determine a blood pressure of the user using the pressure measurements and the vibration measurements.

19. The blood pressure measurement system of claim 18, wherein the smartwatch is configured to cause the display to output an indication of the blood pressure of the user.

20. The blood pressure measurement system of claim 17, wherein:
the first location is along a dorsal side of a wrist of the user; and
the second location is along a volar side of the wrist of the user.
